(19) United States
(12) Patent Application Publication
(10) Pub. No.: US 2010/0256191 A1
(43) Pub. Date: Oct. 7, 2010

(54) AMPA RECEPTOR ANTAGONISTS AND ZONISAMIDE FOR EPILEPSY

(75) Inventors: Takahisa Hanada, Tsukuba-shi (JP); Shigeki Hibi, Ridgefield Park, NJ (US); Kazuki Miyazaki, Ibaraki (JP)

Correspondence Address: CLARK & ELBING LLP, 101 FEDERAL STREET, BOSTON, MA, 02110, US

(73) Assignee: Eisai R&D Management Co., Ltd., Tokyo (JP)

(21) Appl. No.: 12/744,444

(22) PCT Filed: Dec. 26, 2008
(86) PCT No.: PCT/JP2008/073974

§ 371 Date: May 24, 2010

Related U.S. Application Data
(60) Provisional application No. 61/006,133, filed on Dec. 26, 2007.

(51) Int. Cl. A61K 31/444; A61K 31/423; A61P 25/08

(52) U.S. Cl. 514/334; 514/379

(57) ABSTRACT

The invention provides methods for treating epilepsy by administering to patients therapeutically effective amounts of AMPA receptor antagonists in combination with zonisamide useful for treating epilepsy. The invention also provides pharmaceutical combinations, kits, and pharmaceutical compositions comprising therapeutically effective amounts of AMPA receptor antagonists, and optionally, zonisamide that are useful for treating epilepsy.

### RELATED APPLICATION

This application claims priority under 35 U.S.C. §119 to U.S. Provisional Application No. 61/006,133 filed on Dec. 26, 2007, the disclosures of which are incorporated by reference herein in their entirety.

### FIELD OF THE INVENTION

The invention provides pharmaceutical compositions, combinations, and kits comprising AMPA receptor antagonists and zonisamide, and methods using AMPA receptor antagonists and zonisamide for treating epilepsy.

### BACKGROUND OF THE INVENTION

Epilepsy is common neurological disease showing repetitive seizures. Incidence of this disease is around 1% of the total population. They are treated with antiepileptics which mainly modulate voltage sensitive ion channels, GABA receptor or GABA metabolism. Large part of patients respond to the antiepileptic drugs but about 20% of epilepsy patients is refractory to the treatments. Treatment for those refractory epilepsy patients will be add-on therapy. It is recommended to coadminister drugs with different mode of action.

AMPA receptor is distributed throughout brain and has a role in fast synaptic neurotransmission. It is believed that AMPA receptor play a role in abnormal excitation of neuron and neuronal cell death. Broad spectrum anti-seizure effect of AMPA antagonist is confirmed from the studies with various experimental models. Therefore AMPA antagonist could be a good drug candidate for the treatment of epilepsy as monotherapy and adjunctive therapy. However AMPA antagonist has not been authorized as a drug.

AMPA receptor antagonists include 1,2-dihydropyridine compounds. An exemplary 1,2-dihydropyridine compound is perampanel [i.e., 3-(2-cyanophenyl)-5-(2-pyridyl)-1-phenyl-1,2-dihydropyridin-2-one], and is described in U.S. Pat. No. 6,949,571. Methods for treating diseases and administering these compounds in conjunction with a cholinesterase inhibitor are described in WO 2006/107859 and WO 2006/107860. Methods for treating diseases and administering these compounds in conjunction with a NMDA receptor antagonist are described in WO 2008/111590. Methods for treating diseases and administering these compounds in conjunction with a cinnamide compound are described in WO 2008/139984.

Zonisamide [i.e., 3-sulfamoylmethyl-1,2-benzisoxazole and 1,2-benzisoxazole-3-methanesulfonamide; e.g., Merck Index, 12th Ed., 10323 (1996)] is disclosed for its usefulness as an antiepileptic drug for treating various epilepsy seizures (Japanese Examined Application No. 60-33114, Japanese Examined Application No. 61-59288, U.S. Pat. No. 4,172,896, Epilepsy Research 29, 109-114, 1998), for treating ischemic brain disorder (Japanese Examined Application No. 7-84384, U.S. Pat. No. 5,128,354, Brain Research 770, 115-122, 1997), and for treating diseases caused by neurodegeneration such as Parkinson's disease, Huntington's disease, chorea syndrome and distonia syndrome for it shows extremely strong suppressant action to dopaminergic neurodegeneration induced by MPTP (1-methyl-4-phenyl-1,2,3,6-tetrahydropyridine) (Japanese Patent No. 3364481, Neurosci. Res., 41, 397-399, 2001, Current Pharmaceutical Design, 10, 687-693, 2004.).

Zonisamide is the drug developed for the treatment of seizures. This drug mainly ustilized for the treatment of partial seizures. It is also reported that Zonisamide could reduce generalized seizures. Mode of action of Zonisamide is not fully addressed but sodium and calcium channel blockade could explain antiepileptic effect of Zonisamide.

There is a need in the art for treating epilepsy using novel pharmaceutical compositions or combinations. The invention is directed to these, as well as other, important goals.

### SUMMARY OF THE INVENTION

The invention provides methods for treatment and/or prophylaxis of epilepsy in a patient in need thereof by administering a therapeutically effective amount of at least one AMPA receptor antagonist (e.g., 1,2-dihydropyridine compound), optionally in combination with zonisamide or a pharmaceutically acceptable salt thereof. In one embodiment, the AMPA receptor antagonist is 3-(2-cyanophenyl)-5-(2-pyridyl)-1-phenyl-1,2-dihydropyridin-2-one. The methods for the treatment and/or prophylaxis of epilepsy include the treatment and/or prophylaxis of one or more symptoms of epilepsy.

The invention provides methods for treatment and/or prophylaxis of partial seizure or generalized seizure in a patient in need thereof by administering a therapeutically effective amount of at least one AMPA receptor antagonist (e.g., 1,2-dihydropyridine compound), optionally in combination with zonisamide or a pharmaceutically acceptable salt thereof. In one embodiment, the AMPA receptor antagonist is 3-(2-cyanophenyl)-5-(2-pyridyl)-1-phenyl-1,2-dihydropyridin-2-one. The methods for the treatment and/or prophylaxis of partial seizure or generalized seizure include the treatment and/or prophylaxis of one or more symptoms of the partial seizure or generalized seizure.

The invention provides pharmaceutical compositions, combinations, and kits comprising a therapeutically effective amount of at least one AMPA receptor antagonist (e.g., 3-(2-cyanophenyl)-5-(2-pyridyl)-1-phenyl-1,2-dihydropyridin-2-one), and optionally zonisamide or a pharmaceutically acceptable salt thereof.

The invention provides pharmaceutical compositions, combinations, and kits comprising a therapeutically effective amount of: (i) at least one AMPA receptor antagonist (e.g., 3-(2-cyanophenyl)-5-(2-pyridyl)-1-phenyl-1,2-dihydropyridin-2-one), and (ii) optionally zonisamide or a pharmaceutically acceptable salt thereof.

The present invention relates to the following:

(1) A pharmaceutical composition comprising:

(A) an AMPA receptor antagonist, a pharmaceutically acceptable salt thereof, a hydrate thereof, or a hydrate of a pharmaceutically acceptable salt thereof;

(B) zonisamide or a pharmaceutically acceptable salt thereof; and

(C) one or more pharmaceutically acceptable carries.

(2) The pharmaceutical composition of (1), wherein the AMPA receptor antagonist, pharmaceutically acceptable salt thereof, hydrate thereof or the hydrate of the pharmaceutically acceptable salt thereof is a compound of Formula (III), a pharmaceutically acceptable salt thereof, a hydrate thereof, or a hydrate of a pharmaceutically acceptable salt thereof, wherein the compound of Formula (III) is:

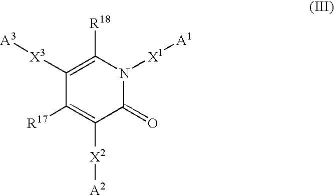

wherein X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶); R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

(3) The pharmaceutical composition of (1), wherein the AMPA receptor antagonist, pharmaceutically acceptable salt thereof, hydrate thereof, or hydrate of a pharmaceutically acceptable salt thereof is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof

(4) The pharmaceutical composition of (1), wherein the composition is used for treating epilepsy or one or more symptoms of epilepsy.

(5) The pharmaceutical composition of (1), wherein the composition is used for treating partial seizure or one or more symptoms of partial seizure.

(6) The pharmaceutical composition of (5), wherein the partial seizure is a simple partial seizure, complex partial seizure, or a partial seizure secondarily generalized.

(7) The pharmaceutical composition of (1), wherein the composition is used for treating generalized seizure or one or more symptoms of generalized seizure.

(8) The pharmaceutical composition of (7), wherein the generalized seizure is an absence seizures, myoclonic seizures, clonic seizures, tonic seizures, tonic-clonic seizures or atonic seizures.

(9) The pharmaceutical composition of (1), wherein the composition is adapted to be associated with a treatment regimen.

(10) A combination comprising:

(A) an AMPA receptor antagonist, a pharmaceutically acceptable salt thereof, a hydrate thereof, or a hydrate of a pharmaceutically acceptable salt thereof; and

(B) zonisamide or a pharmaceutically acceptable salt thereof.

(11) The combination of (10), wherein the AMPA receptor antagonist, pharmaceutically acceptable salt thereof, hydrate thereof, or the hydrate of the pharmaceutically acceptable salt thereof is a compound of Formula (III), a pharmaceutically acceptable salt thereof, a hydrate thereof, or a hydrate of a pharmaceutically acceptable salt thereof; wherein the compound of Formula (III) is:

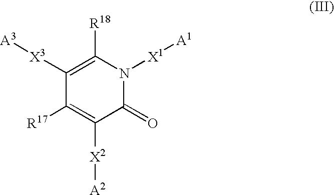

wherein X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶), R⁶, R⁷, R⁸, R⁹, R₁₀, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

(12) The combination of (10), wherein the AIVIF′A receptor antagonist, pharmaceutically acceptable salt thereof, hydrate thereof, or hydrate of a pharmaceutically acceptable salt thereof is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof

(13) The combination of (10), wherein (A) and (B) are administered separately to a patient or are administered to a patient in the form of a pharmaceutical composition.

(14) The combination of (10), wherein the combination is used for treating epilepsy or one or more symptoms of epilepsy.

(15) The combination of (10), wherein the combination is used for treating partial seizure or one or more symptoms of partial seizure.

(16) The combination of (15), wherein the partial seizure is a simple partial seizure, complex partial seizure, or a partial seizure secondarily generalized.

(17) The combination of (10), wherein the combination is used for treating generalized seizure or one or more symptoms of generalized seizure.

(18) The combination of (17), wherein the generalized seizure is an absence seizures, myoclonic seizures, clonic seizures, tonic seizures, tonic-clonic seizures or atonic seizures.

(19) The combination of (10), wherein the combination is adapted to be associated with a treatment regimen.

(20) Use of compounds (A) and (B) for producing a pharmaceutical composition in the treatment of epilepsy or one or more symptoms of epilepsy, wherein (A) and (B) are:

(A) an AMPA receptor antagonist, a pharmaceutically acceptable salt thereof, a hydrate thereof, or a hydrate of a pharmaceutically acceptable salt thereof, and

(B) zonisamide or a pharmaceutically acceptable salt thereof (21) Use of compounds (A) and (B) for producing a pharmaceutical composition in the treatment of partial seizure or one or more symptoms of partial seizure, wherein (A) and (B) are:

(A) an AMPA receptor antagonist, a pharmaceutically acceptable salt thereof, a hydrate thereof, or a hydrate of a pharmaceutically acceptable salt thereof, and

(B) zonisamide or a pharmaceutically acceptable salt thereof.

(22) The use of (21), wherein the partial seizure is a simple partial seizure, complex partial seizure, or a partial seizure secondarily generalized.

(23) Use of compounds (A) and (B) for producing a pharmaceutical composition in the treatment of generalized seizure or one or more symptoms of generalized seizure, wherein (A) and (B) are:

(A) an AMPA receptor antagonist, a pharmaceutically acceptable salt thereof, a hydrate thereof, or a hydrate of a pharmaceutically acceptable salt thereof; and

(B) zonisamide or a pharmaceutically acceptable salt thereof.

(24) The use of (23), wherein the generalized seizure is an absence seizures, myoclonic seizures, clonic seizures, tonic seizures, tonic-clonic seizures or atonic seizures.

(25) The use of any one of (20) to (24), wherein the AMPA receptor antagonist, pharmaceutically acceptable salt thereof, hydrate thereof, or the hydrate of the pharmaceutically acceptable salt thereof is a compound of Formula (III), a pharmaceutically acceptable salt thereof, a hydrate thereof, or a hydrate of a pharmaceutically acceptable salt thereof, wherein the compound of Formula (III) is:

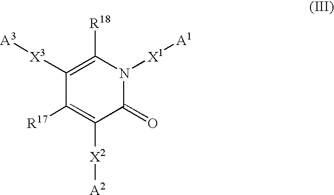

wherein X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —N(R⁶)—, —N(R⁷)—CO—, —CO—N(R⁸)—, —N(R⁹)—CH₂—, —CH₂—N(R¹⁰)—, —CH₂—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶), R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

(26) The use of any one of (20) to (24), wherein the AMPA receptor antagonist, pharmaceutically acceptable salt thereof, hydrate thereof, or hydrate of a pharmaceutically acceptable salt thereof is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof

(27) The use of any one of (20) to (24), wherein (A) and (B) are administered separately to a patient or are administered to a patient in the form of a pharmaceutical composition.

(28) The use of any one of (20) to (24), wherein the treatment is part of a treatment regimen.

(29) Compounds (A) and (B) for use in the treatment of epilepsy or one or more symptoms of epilepsy, wherein (A) and (B) are:

(A) an AMPA receptor antagonist, a pharmaceutically acceptable salt thereof, a hydrate thereof, or a hydrate of a pharmaceutically acceptable salt thereof; and

(B) zonisamide or a pharmaceutically acceptable salt thereof.

(30) Compounds (A) and (B) for use in the treatment of partial seizure or one or more symptoms of partial seizure, wherein (A) and (B) are:

(A) an AMPA receptor antagonist, a pharmaceutically acceptable salt thereof, a hydrate thereof, or a hydrate of a pharmaceutically acceptable salt thereof, and

(B) zonisamide or a pharmaceutically acceptable salt thereof.

(31) The compound of (30), wherein the partial seizure is a simple partial seizure, complex partial seizure, or a partial seizure secondarily generalized.

(32) Compounds (A) and (B) for use in the treatment of generalized seizure or one or more symptoms of generalized seizure, wherein (A) and (B) are:

(A) an AMPA receptor antagonist, a pharmaceutically acceptable salt thereof, a hydrate thereof, or a hydrate of a pharmaceutically acceptable salt thereof; and

(B) zonisamide or a pharmaceutically acceptable salt thereof.

(33) The compound of (32), wherein the generalized seizure is an absence seizures, myoclonic seizures, clonic seizures, tonic seizures, tonic-clonic seizures or atonic seizures.

(34) The compound of any one of (29) to (33), wherein the treatment is part of a treatment regimen.

(35) A kit comprising the pharmaceutical composition of any one of (1) to (9) or the combination of any one of (10) to (19).

(36) The kit of (35), wherein the kit is adapted to be associated with a treatment regimen.

(37) A method for treating epilepsy or one or more symptoms of epilepsy comprising administering to a patient in need thereof a therapeutically effective amount of the pharmaceutical composition of any one of (1) to (9) or a therapeutically effective amount of the combination of any one of (10) to (19).

(38) A method for treating partial seizure or one or more symptoms of partial seizure comprising administering to a patient in need thereof a therapeutically effective amount of the pharmaceutical composition of any one of (1) to (9) or a therapeutically effective amount of the combination of any one of (10) to (19).

(39) The method of (38), wherein the partial seizure is a simple partial seizure, complex partial seizure, or a partial seizure secondarily generalized.

(40) A method for treating generalized seizure or one or more symptoms of generalized seizure comprising administering to a patient in need thereof a therapeutically effective amount of the pharmaceutical composition of any one of (1) to (9) or a therapeutically effective amount of the combination of any one of (10) to (19).

(41) The method of (40), wherein the generalized seizure is an absence seizures, myoclonic seizures, clonic seizures, tonic seizures, tonic-clonic seizures or atonic seizures.

(42) The method of any one of (37) to (41), wherein the treatment is part of a treatment regimen and the administration involves a series of administrations.

These and other aspects of the invention are described in more detail herein.

### DETAILED DESCRIPTION OF THE INVENTION

“Patient” refers to animals, preferably mammals, more preferably humans. The term “patient” includes men and women; and includes adults, children and neonates. In one embodiment, the patient can be an animal companion, such as a dog or a cat.

“Active ingredient” refers to the AMPA receptor antagonists and zonisamide, described herein, that are responsible for treatment and/or prophylaxis of a disease or disorder. The active ingredients may have mechanisms of action that are known or unknown, and the active ingredients may have one or more mechanisms of action. The active ingredient may have an asymmetric carbon depending on the type of substituent and may have a stereoisomer (e.g., a geometric isomer, an enantiomer, a diastereomer or the like). The active ingredient or a stereoisomer thereof may form a pharmaceutically acceptable salt. The active ingredient, a pharmaceutically acceptable salt thereof, a stereoisomer thereof or a pharmaceutically acceptable salt of a stereoisomer thereof may be an anhydride, and may form a solvate. The active ingredient, a pharmaceutically acceptable salt thereof, a stereoisomer thereof, a pharmaceutically acceptable salt of a stereoisomer thereof or a solvate thereof may be crystalline or amorphous. Crystalline polymorphs may exist in the active ingredient, a pharmaceutically acceptable salt thereof, a stereoisomer thereof, a pharmaceutically acceptable salt of a stereoisomer thereof or a solvate thereof, although not limited thereto and any form of crystal may exist alone or in combination, which are within the scope of the present invention.

“Treatment” and “treating” refer to the acquisition of a desired pharmacological effect and/or physiologic effect. These effects are prophylactic in terms of completely or partially preventing a disease and/or one or more symptom(s) of the disease, and therapeutic in terms of partially or completely curing a disease and/or one or more symptoms caused by a disease. “Treatment” and “treating” include any treatment of a disease (e.g., epilepsy) in a patient including, for example: (a) to prevent a disease or one or more symptom(s) of the disease in a patient who is suspected of being predisposed to the disease but not yet been diagnosed as having the disease or who has previously been diagnosed as having the disease but is not currently diagnosed as having the disease; (b) to inhibit one or more symptom(s) of a disease, i.e., to inhibit or delay the progression of one or more of the symptom(s) of the disease; (c) to alleviate one or more symptom(s) of a disease, i.e., to reverse or eliminate one or more symptom(s) of the disease; (d) to reverse the progress of one or more symptom(s) of the disease; or (e) to stabilize one or more symptom(s) of a disease, such that one or more symptom(s) of the disease do not worsen or improve. A particular treatment regimen for a patient can depend upon a variety of factors, including the activity of the specific compound employed, the age, body weight, general health, sex, diet, time of administration, rate of excretion, drug combination, and the judgment of the treating physician and the severity of the condition treated.

“Administered separately” with reference to the administration of two or more compounds to treat and/or prevent and/or delay the onset of the diseases and disorders described herein includes, for example, the sequential administration of the compounds in any order or the simultaneous administration of the compounds. Simultaneous administration of the compounds means that the compounds are administered to the patient at substantially the same time or at exactly the same time, depending on the mode of administration. The sequential administration of the compounds may occur in any order and may occur with any amount of time elapsing between administrations of the compounds. Sequential administration may be based on factors that would influence which of the compounds should be administered first and which should be administered second, and how much time should elapse between administrations of the compounds. For example, when two or more compounds are administered separately and sequentially, factors that effect when the compounds are administered to the patient include, for the time(s) that provides the fewest side effects for the compound being administered, (c) the dosage of the compound, (d) the route of administration of the compound, (e) the disease or disorder being treated, (f) the patient being treated, (g) the in vivo relationship of the compounds being administered, and other such factors known in the art. Preferably, the time intervals for sequential administration are chosen so that the effect on the disease or disorder being treated in the combined use of the active ingredients is greater than additive when compared to the effect which would be obtained by use of only one of the active ingredients.

“Combination” refers to the AMPA receptor antagonist and the second active ingredient (e.g., zonisamide) being administered separately as distinct pharmaceutical compositions or formulations (e.g., a first pharmaceutical composition comprising a AMPA receptor antagonist and a second pharmaceutical composition comprising zonisamide). The pharmaceutical compositions or formulations can have the same or different modes of administration.

“Monotherapy” is a therapy which uses only one active ingredient (e.g., an AMPA receptor antagonist) for treatment and/or prophylaxis of a disease or disorder.

“Combination therapy” is a therapy where two or more active ingredients are administered separately or are administered in the form of a pharmaceutical composition for the treatment and/or prophylaxis of a disease.

“Therapeutically effective amount” refers to the amount of the active ingredient that is necessary for the treatment and/or prophylaxis of a disease. When two or more active ingredients are administered for combination therapy, the term “therapeutically effective amount” refers to the amount of active ingredients that are necessary for treatment and/or prophylaxis of a disease and includes, for example: (a) a therapeutically effective amount of a first active ingredient and a therapeutically effective amount of a second active ingredient (i.e., the amount of each active ingredient that would be used for monotherapy for the treatment and/or prophylaxis of a disease is used for the combination therapy); (b) a therapeutically effective amount of a first active ingredient and a sub-therapeutic amount of a second active ingredient, which in combination effectively provide for treatment and/or prophylaxis of a disease (e.g., the sub-therapeutic amount of the second active ingredient can be used in combination therapy to achieve a result that would be equal to or greater than the result that the second active ingredient would achieve if it was used for monotherapy); (c) a sub-therapeutic amount of a first active ingredient and a therapeutically effective amount of a second active ingredient, which in combination effectively provide for treatment and/or prophylaxis of a disease (e.g., the sub-therapeutic amount of the first active ingredient can be used in combination therapy to achieve a result that would be equal to or greater than the result that the first active ingredient would achieve if it was used for monotherapy); and (d) a sub-therapeutic amount of a first active ingredient and a sub-therapeutic amount of a second active ingredient, which in combination therapy provide for treatment and/or prophylaxis of a disease or disorder (e.g., the sub-therapeutic amount of the first active ingredient can be used in combination therapy to achieve a result that would be equal to or greater than the result that the first active ingredient would achieve if it was used for monotherapy; and the sub-therapeutic amount of the second active ingredient can be used in combination therapy to achieve a result that would be equal to or greater than the result that the second active ingredient would achieve if it was used for monotherapy). The same therapeutic/sub-therapeutic amounts can be used when there are three or more active ingredients used in combination therapy. For example, (a) there may be therapeutically effective amounts of all three active ingredients; (b) there may be therapeutically effective amounts of two active ingredients and a sub-therapeutic amount of a third active ingredient; (c) there may be a therapeutically effective amount of one active ingredient and sub-therapeutic amounts of two other active ingredients; or (d) there may be sub-therapeutic amounts of all three active ingredients.

“Kits,” also known as commercial packages, can include a combination of (i) a first pharmaceutical composition or formulation comprising the AMPA receptor antagonist; (ii) a second pharmaceutical composition or formulation comprising the second active ingredient (e.g., zonisamide); (iii) instructions for using the pharmaceutical compositions or formulations for treating or preventing or delaying the onset of the disease; and (iv) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The kit can supply enough medication and materials for days, weeks or months. In another embodiment, “kits” can include (i) pharmaceutical composition or formulation comprising both the AMPA receptor antagonist and the second active ingredient (e.g., zonisamide); (ii) instructions for using the pharmaceutical composition or formulation for treating or preventing or delaying the onset of the disease; and (iii) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The kit can supply enough medication and materials for days, weeks or months. Additionally, the kit can supply enough medication and materials to complete all or a portion of a treatment regimen.

“Solvate” is well known in the art. The solvate is preferably a pharmaceutically acceptable solvate. The pharmaceutically acceptable solvate may be either a hydrate or a nonhydrate, but preferably a hydrate. The solvent such as water, alcohol (e.g., methanol, ethanol, n-propanol), dimethylformamide, dimethyl sulfoxide (DMSO) or the like may be used.

“Hydrate” refers to a compound containing a molecule of water of crystallization. The molecule of water of crystallization can be an integer of 1 or more, such as 1 to 10; or can be any fraction greater than 0 or a fraction of an integer from 1 to 10. For example, the hydrate may be represented as compound.¼H₂O; compound.½H₂O; compound.¾H₂O; compound.2H₂O; compound.5½H₂O; compound.6H₂O; and the like. The “compound” can be any described herein, such as 3-(2-cyanophenyl)-5-(2-pyridyl)-1-phenyl-1,2-dihydropyridin-2-one.

“Pharmaceutically acceptable salts” are well known in the art and include those of inorganic acids, such as hydrochloride, sulfate, hydrobromide and phosphate; and those of organic acids, such as formate, tartrate, acetate, trifluoroacetate, methanesulfonate, benzenesulfonate and toluenesulfonate. When certain substituents are selected, the compounds of the invention can form, for example, alkali metal salts, such as sodium or potassium salts; alkaline earth metal salts, such as calcium or magnesium salts; organic amine salts, such as a salt with trimethyl-amine, triethylamine, pyridine, picoline, dicyclohexylamine or N,N′-dibenzylethylenediamine. One skilled in the art will recognize that the compounds of the invention can be made in the form of any other pharmaceutically acceptable salt.

The invention provides methods for the treatment and/or prophylaxis of epilepsy using at least one AMPA receptor antagonist and, optionally, zonisamide or a pharmaceutically acceptable salt thereof.

### I. Epilepsy

For the purposes of clinical assessment, patients are classified according to the type of seizure. There are two classes of seizures: partial seizures and generalized seizures. Partial seizures are further classified as simple partial seizures, complex partial seizures and partial seizures secondarily generalized. Generalized seizures are classified as convulsive and nonconvulsive seizures. They are further classified as absence (previously referred to as ‘petit mal’) seizures, atypical absence seizures, myoclonic seizures, clonic seizures, tonic seizures, tonic-clonic seizures, and atonic seizures.

(1) Partial seizure can be classified into the following three fundamental groups.
A. Simple partial seizure
B. Complex partial seizure
C. Partial seizures secondarily generalized

(2) Generalized seizures involve bilateral hemispheres in the first clinical change. Following types of seizures are included in the generalized seizures.
A. Absence seizures
   i) Absence seizure
   ii) Atypical absence seizures
B. Myoclonic seizures
C. Clonic seizures
D. Tonic seizures
E. Tonic-clonic seizures
F. Atonic seizures.

(3) Unclassified epileptic seizures

Unclassified epileptic seizures include all seizures that can not be classified into the above listed seizures (1) and (2) because of inadequate or incomplete clinical data, and because of some causes that were not defined hitherto into the described categories. Examples of the unclassified epileptic seizures include some of neonatal seizures, e.g. rhythmic eye movements, chewing, and swimming.
Detail of classification of epilepsy or epileptic syndrome is described in the art (Epilepsia (1989) 30; 389-399) which is incorporated herein in their entirety for reference.
Following diseases (1) to (3) are diagnosed in accordance with the combination of the above classified or unclassified seizures.

(1) Idiopathic epilepsies
A. Idiopathic epilepsies with partial seizures
B. Idiopathic epilepsies with generalized seizures

(2) Symptomatic epilepsies
A. Symptomatic epilepsies with partial seizures
B. Symptomatic epilepsies with generalized seizures

(3) Epilepsies that are difficult to categorize
A. West's syndrome
B. Lennox-Gastaut syndrome
C. Acquired epileptic aphasia (the Landau-Kleffner syndrome)
D. Epilepsy with continuous spike-wave during slow wave sleep

“Epilepsy” refers to a brain disorder in which clusters of nerve cells, or neurons, in the brain sometimes signal abnormally. In epilepsy, the normal pattern of neuronal activity becomes disturbed, causing strange sensations, emotions and behavior, or sometimes convulsions, muscle spasms, and loss of consciousness. Only when a person has had two or more seizures is she or he considered to have epilepsy.

“Epilepsy” can be classified as partial seizure generalized seizure or unclassified epileptic seizures (adapted from Epilepsia (1981) 22; 489-501).

“Epilepsy syndrome,” refers to disorders characterized by a specific set of symptoms that include epilepsy. Epilepsy syndromes are described by their symptoms or by where in the brain they originate.

“Partial seizure” refers to focal seizures that occur in just one part of the brain.

“Simple partial seizure” refers to focal seizures that occur in just one part of the brain where the person experiencing the focal seizure remains conscious but may experience unusual feelings or sensations.

“Complex partial seizure” refers to a focal seizures where the person suffers a change in or loss of consciousness.

“Partial seizures secondarily generalized” refer to seizures which start as a partial seizure, i.e., they start in one limited area of the brain and then spread throughout the brain, becoming “generalized.”
“Absence seizure” refers to a epilepsy disorder where there are repeated instances of seizures where there are momentary losses of consciousness. These seizures almost always start in childhood or adolescence.

“Atypical absence” seizures refer absence seizures with atypic features, e.g. bizarre motor activity.

“Myoclonic seizures” refer to seizures that cause sudden jerks or twitches, especially in the upper body, arms or legs.

“Clonic seizures” refer to seizures that cause repeated jerking movements of muscles on both sides of the body.

“Tonic seizures” refer to seizures that cause stiffening of muscles of the body, generally those in the back, legs and arms.

“Tonic-clonic seizures” (“grand mal seizures”) refer to seizures that cause a mixture of symptoms, including loss of consciousness, stiffening of the body, and repeated jerks of the arms and legs.

“Atonic seizures” (“drop attacks”) refer to seizures that cause a sudden loss of muscle tone.

In one embodiment, the AMPA receptor antagonist used in the methods and compositions described herein may be any known in the art. Exemplary AMPA receptor antagonists, all of which are active ingredients, include 1,2-dihydropyridine compounds, quinoxalinedione aminoalkylphosphonates, and the like.

In one embodiment, the AMPA receptor antagonist may be becampanel, EGIS 8332 (7-acetyl-5-(4-aminophenyl)-8,9-dihydro-8-methyl-7H-1,3-dioxolo[4,5-h][2,3]benzodiazepine-8-carbonitrile); GYKI 47261 (4-(7-chloro-2-methyl-4H-3,10,10a-triaza-benzo[f]azulen-9-yl)phenylamine)); irampanel (N,N-dimethyl-2-[2-(3-phenyl-1,2,4-oxadiazol-5-yl)phenoxy]ethanamine); KRP 199 ((7-[4-[[[[4-carboxyphenyl)-amino]carbonyl]oxy]methyl]-1H-imidazol-1-yl]-3,4-dihydro-3-oxo-6-(trifluoromethyl)-2-quinoxalinecarboxylic acid); NS 1209 (2-[[[5-[4-[(dimethylamino)-sulfonyl]phenyl]-1,2,6,7,8,9-hexahydro-8-methyl-2-oxo-3H-pyrrolo[3,2-h]isoquinolin-3-ylidene]amino]oxy]-4-hydroxybutanoic acid monosodium salt; topiramate (TOPAMAX®); talampanel (LY-300164, (R)-7-acetyl-5-(4-aminophenyl)-8,9-dihydro-8 methyl-7H-1,3-dioxolo[4,5-h][2,3]benzo-diazepine; YM9OK (6-imidazol-1-yl-7-nitro-1,4-dihydro-quinoxaline-2,3-dione); S-34730 (7-chloro-6-sulfamoyl-2-(1H)-quinolinone-3-phosphonic acid); Zonampanel (YM-872; (7-imidazol-1-yl-6-nitro-2,3-dioxo-3,4-dihydro-2H-quinoxalin-1-yl)-acetic acid); GYM 52466 (4-(8-methyl-9H-1,3-dioxa-6,7-diaza-cyclohepta[f]inden-5-yl)-phenylamine); ZK 200775 (MPQX, (7-morpholin-4-yl-2,3-dioxo-6-trifluoromethyl-3,4-dihydro-2H-quinoxalin-1-ylmethyl)-phosphonic acid); CP-465022 (3-(2-chlorophenyl)-2-[2-(6-diethylaminomethyl-pyridin-2-yl)-vinyl]-6-fluoro-3H-quinazolin-4-one); SYM-2189 (4-(4-amino-phenyl)-6-methoxy-1-methyl-1H-phthalazine-2-carboxylic acid propylamide); SYM-2206 (8-(4-amino-phenyl)-5-methyl-5H-[1,3]dioxolo[4,5-g]phthalazine-6-carboxylic acid propylamide); RPR-117824 ((4-oxo-2-phosphono-5,10-dihydro-4H-imidazo[1,2-a]indeno[1,2-e]pyrazin-9-yl)-acetic acid); or LY-293558 (6-[2-(1H-tetrazol-5-yl)-ethyl]-decahydro-isoquinoline-3-carboxylic acid).

In another embodiment, the AMPA receptor antagonist is a 1,2-dihydropyridine compound. The 1,2-dihydropyridine compound used in the methods and compositions described herein may be any known in the art. The term “1,2-dihydropyridine compound” includes 1,2-dihydropyridine compounds, pharmaceutically acceptable salts of 1,2-dihydropyridine compounds, stereoisomers of 1,2-dihydropyridine compounds, pharmaceutically acceptable salts of stereoisomers of 1,2-dihydropyridine compounds, hydrates of 1,2-dihydropyridine compounds, hydrates of pharmaceutically acceptable salts of 1,2-dihydropyridine compounds, stereoisomers of hydrates of 1,2-dihydropyridine compounds, and stereoisomer of hydrates of pharmaceutically acceptable salts of 1,2-dihydropyridine compounds.

The 1,2-dihydropyridine compound used in the methods and compositions described herein may be a compound of Formula (I):

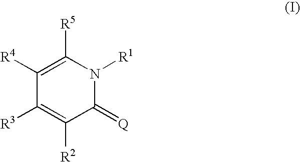

wherein

Q is NH, O or S;

R¹, R², R³, R⁴ and R⁵ are each independently hydrogen, halogen, C₁₋₆ alkyl, or —X-A;

X is a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶)—;

R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy;

m, n, p and q are each independently an integer of 0, 1 or 2;

A is an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5- to 14-membered aromatic heterocyclic ring; provided that 3 groups among R¹, R², R³, R⁴ and R⁵ are —X-A; and that the residual 2 groups among R¹, R², R³, R⁴ and R⁵ are independently hydrogen, halogen, or C₁₋₆ alkyl.

In one embodiment, the following compounds are excluded from the scope of the compound of Formula (I): (1) when Q is O; R¹ and R⁵ are hydrogen; and R², R³ and R⁴ are phenyl; (2) when Q is O; R¹ and R⁴ are hydrogen; and R², R³ and R⁵ are phenyl; and (3) when Q is O; R¹ and R² are hydrogen; and R³, R⁴ and R⁵ are phenyl.

In another embodiment, the 1,2-dihydropyridine compound used in the methods and compositions described herein is a compound of Formula (II):

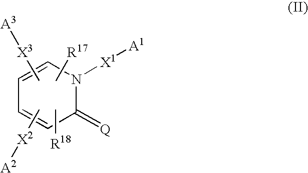

wherein

Q is NH, O or S;

X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶);

R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy;

m, n, p and q are each independently an integer of 0, 1 or 2;

A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and

R¹⁷ and R¹⁸ are each independently hydrogen, halogen, or C₁₋₆ alkyl.

In another embodiment, the invention provides the compound of Formula (II) wherein X¹, X² and X³ are each independently a single bond, an optionally substituted C₁₋₆ alkylene, an optionally substituted C₂₋₆ alkenylene, or an optionally substituted C₂₋₆ alkynylene. The substituents may be one or more of —O—, —S—, —CO—, —SO—, —SO₂—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— and —N(R¹⁵)—CS—N(R¹⁶)—;

R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy;

m, n, p and q are each independently an integer of 0, 1 or 2;

A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5- to 14-membered aromatic heterocyclic ring.

The substituents for the 1,2-dihydropyridine compounds of the invention may be one or more of hydroxy; halogen; nitrile; nitro; C₁₋₆ alkyl; C₂₋₆ alkenyl; C₂₋₆ alkynyl [wherein the alkyl, alkenyl, and alkynyl can independently and optionally be substituted with one or more groups selected from hydroxy, nitrile, halogen, C₁₋₆ alkylamino, di(C₁₋₆ alkyl)amino, C₂₋₆ alkenylamino, di(C₂₋₆ alkenyl)amino, C₂₋₆ alkynylamino, di(C₂₋₆ alkynyl)amino, alkyl-N—C₂₋₆ alkenylamino, N—C₁₋₆ alkyl-N—C₂₋₆ alkynylamino, N—C₂₋₆alkenyl-N—C₂₋₆alkynylamino, aralkyloxy, TBDMS oxy, C₁₋₆ alkylsulfonylamino, C₁₋₆ alkylcarbonyloxy, C₂₋₆ alkenylcarbonyloxy, C₂₋₆ alkynylcarbonyloxy, N—C₁₋₆ alkylcarbamoyl, alkenylcarbamoyl, and N—C₁₋₆ alkynylcarbamoyl]; C₁₋₆ alkoxy; C₂₋₆ alkenyloxy; C₂₋₆ alkynyloxy [wherein the alkoxy, alkenyloxy, and alkynyloxy may independently and optionally be substituted with one or more groups selected from C₁₋₆ alkylamino, aralkyloxy, and hydroxy]; C₁₋₆ alkylthio; C₂₋₆ alkenylthio; C₂₋₆ alkynylthio [wherein the alkylthio, alkenylthio, and alkynylthio may independently and optionally be substituted with one or more groups selected from hydroxy, nitrile, halogen, C₁₋₆ alkylamino, aralkyloxy, TBDMS oxy, C₁₋₆ alkylsulfonylamino, C₁₋₆ alkylcarbonyloxy, and C₁₋₆ alkylcarbamoyl]; optionally substituted carbonyl [which may be substituted with C₁₋₆ alkoxy, amino, C₁₋₆ alkylamino, di(C₁₋₆ alkylamino, C₂₋₆ alkenylamino, di(C₂₋₆ alkenyl)amino, C₂₋₆ alkynylamino, di(C₂₋₆ alkynyl)amino, N—C₁₋₆alkyl-N—C₂₋₆ alkenylamino, alkyl-N—C₂₋₆ alkynylamino and N—C₂₋₆ alkenyl-N—C₂₋₆ alkynylamino]; an optionally substituted amino [which may be substituted with one or two groups selected from C₁₋₆ alkyl, C₂₋₆ alkenyl, C₂₋₆ alkynyl, C₁₋₆ alkylsulfonyl, C₂₋₆ alkenylsulfonyl, C₂₋₆ alkynylsulfonyl, C₁₋₆ alkylcarbonyl, C₂₋₆ alkenylcarbonyl and C₂₋₆ alkynylcarbonyl]; C₁₋₆ alkylsulfonyl; C₂₋₆ alkenylsulfonyl; C₂₋₆ alkynylsulfonyl; C₁₋₆alkylsulfinyl; C₂₋₆ alkenylsulfinyl; C₂₋₆ alkynylsulfinyl; formyl; optionally substituted C₃₋₈ cycloalkyl; an optionally substituted C₃₋₈ cycloalkenyl [where the cycloalkyl group and/or the cycloalkenyl group may independently and optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; a 5- to 14-membered non-aromatic heterocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; C₆₋₁₄ aromatic hydrocarbocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; and a 5- to 14-membered aromatic heterocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl].

In another embodiment, the invention provides compounds of Formula (II) wherein A¹, A² and A³ are each independently an optionally substituted C₃₋₈ cycloalkyl, an optionally substituted C₃₋₈ cycloalkenyl or an optionally substituted 5- to 14-membered non-aromatic hetero ring. In another embodiment, the invention provides the compound of Formula (II) wherein A¹, A² and A³ are each independently an optionally substituted C₆₋₁₄ aromatic hydrocarbon ring or an optionally substituted 5- to 14-membered aromatic hetero ring. In another embodiment, the invention provides the compound of Formula (II) wherein A¹, A² and A³ are each independently phenyl, pyrrolyl, pyridyl, pyridazinyl, pyrimidinyl, pyrazinyl, thienyl, thiazolyl, furyl, naphthyl, quinolyl, iso-quinolyl, indolyl, benzimidazolyl, benzothiazolyl, benzoxazolyl, imidazopyridyl, carbazolyl, cyclopentyl, cyclohexyl, cyclohexenyl, dioxinyl, adamantyl, pyrrolidinyl, piperidinyl, piperazinyl or morpholyl; any of which may optionally have substituents. In another embodiment, the invention provides the compound of Formula (II) wherein A¹, A² and A³ are each independently selected from:

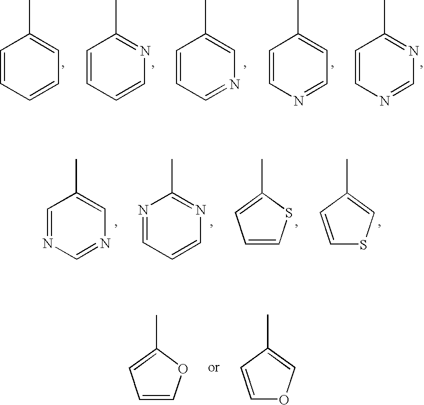

each of which may optionally be substituted. In another embodiment, the invention provides the compound of Formula (II) wherein A¹, A² and A³ are each independently substituted with hydroxyl, halogen, amino, or nitrile. In another embodiment, the invention provides the compound of Formula (II) wherein A¹, A² and A³ are each independently hydroxyl, halogen, amino, nitrile, or nitro. In another embodiment, the invention provides the compound of Formula (II) wherein Q is oxygen.

In another embodiment, the invention provides the compounds of Formula (I) or (II) wherein X¹, X² and X³ are each independently a single bond, —CH₂—, —CH(OH)—, —CH₂—CH₂—, —CH═CH—, —C≡C—, —O— or —CO—. In another embodiment, the invention provides the compounds of Formula (I) or (II) wherein X¹, X² and X³ are each a single bond. In another embodiment, the invention provides the compounds of Formula (I) or (II) wherein R¹⁷ and R¹⁸ are each independently hydrogen, fluorine, chlorine, bromine, iodine, methyl, ethyl, n-propyl, or iso-propyl. In another embodiment, the invention provides the compounds of Formula (I) or (II) wherein R¹⁷ and R¹⁸ are each hydrogen.

With respect to the 1,2-dihydropyridine compounds of the invention, the halogen atom indicates fluorine, chlorine, bromine, iodine and the like, and the preferable atoms include fluorine, chlorine and bromine.

With respect to the 1,2-dihydropyridine compounds of the invention, the C₁₋₆ alkyl indicates an alkyl having 1 to 6 carbons, and examples include linear chain or branched chain alkyl groups such as methyl, ethyl, n-propyl, iso-propyl, n-butyl, iso-butyl, sec-butyl, tert-butyl, n-pentyl, 1,1-dimethylpropyl, 1,2-dimethylpropyl, 2,2-dimethylpropyl, 1-ethylpropyl, 2-ethylpropyl, n-hexyl, 1-methyl-2-ethylpropyl, 1-ethyl-2-methylpropyl, 1,1,2-trimethylpropyl, 1-propylpropyl, 1-methylbutyl, 2-methylbutyl, 1,1-dimethylbutyl, 1,2-dimethylbutyl, 2,2-dimethylbutyl, 1,3-dimethylbutyl, 2,3-dimethylbutyl, 2-ethylbutyl, 2-methylpentyl, 3-methylpentyl, and the like.

With respect to the 1,2-dihydropyridine compounds of the invention, the C₂₋₆ alkenyl indicates an alkenyl group having 2 to 6 carbons, and examples include vinyl, allyl, 1-propenyl, 2-propenyl, iso-propenyl, 2-methyl-1-propenyl, 3-methyl-1-propenyl, 2-methyl-2-propenyl, 3-methyl-2-propenyl, 1-butenyl, 2-butenyl, 3-butenyl, 1-pentenyl, 1-hexenyl, 1,3-hexadienyl, 1,6-hexadienyl, and the like.

With respect to the 1,2-dihydropyridine compounds of the invention, the C₂₋₆ alkynyl indicates an alkynyl group having 2 to 6 carbons, and examples include ethynyl, 1-propynyl, 2-propynyl, 1-butynyl, 2-butynyl, 3-butynyl, 3-methyl-1-propynyl, 1-ethynyl-2-propynyl, 2-methyl-3-propynyl, 1-pentynyl, 1-hexynyl, 1,3-hexadiynyl, 1,6-hexadiynyl, and the like.

With respect to the 1,2-dihydropyridine compounds of the invention, the C₁₋₆ alkoxy indicates an alkoxy group having 1 to 6 carbons, and examples include methoxy, ethoxy, n-propoxy, iso-propoxy, sec-propoxy, n-butoxy, iso-butoxy, sec-butoxy, tert-butoxy, n-pentyloxy, iso-pentyloxy, sec-pentyloxy, n-hexoxy, iso-hexoxy, 1,1-dimethylpropoxy, 1,2-dimethylpropoxy, 2,2-dimethylpropoxy, 2-ethylpropoxy, 1-methyl-2-ethylpropoxy, 1-ethyl-2-methylpropoxy, 1,1,2-trimethylpropoxy, 1,1,2-trimethylpropoxy, 1,1-dimethylbutoxy, 1,2-dimethylbutoxy, 2,2-dimethylbutoxy, 2,3-dimethylbutoxy, 1,3-dimethylbutoxy, 2-ethylbutoxy, 1,3-dimethylbutoxy, 2-methylpentoxy, 3-methylpentoxy, hexyloxy, and the like.

With respect to the 1,2-dihydropyridine compounds of the invention, the C₂₋₆ alkynyloxy indicates an alkynyloxy group having 2 to 6 carbon atoms, and examples include ethynyloxy, 1-propynyloxy, 2-propynyloxy, 1-butynyloxy, 2-butynyloxy, 3-butynyloxy, 1-methyl-2-propynyloxy, 1-ethyl-2-propynyloxy, 1-ethynyl-2-propynyloxy, 1-pentynyloxy, 1-hexynyloxy, 1,3-hexadiynyloxy, 1,6-hexadiynyloxy, and the like.

With respect to the 1,2-dihydropyridine compounds of the invention, the C₂₋₆ alkenyloxy indicates an alkenyloxy group having 2 to 6 carbons, and examples include vinyloxy, 2-propenyloxy, 1-propenyloxy, 2-propenyloxy, iso-propenyloxy, 2-methyl-1-propenyloxy, 3-methyl-1-propenyloxy, 2-methyl-2-propenyloxy, 3-methyl-2-propenyloxy, 1-butenyloxy, 2-butenyloxy, 3-butenyloxy, 1-pentenyloxy, 1-hexenyloxy, 1,3-hexadienyloxy, 1,6-hexadienyloxy, and the like.

With respect to the 1,2-dihydropyridine compounds of the invention, the C₃₋₈ cycloalkyl indicates a cycloalkyl group composed of 3 to 8 carbon atoms, and examples include cyclopropyl, cyclobutyl, cyclopentyl, cyclohexyl, cycloheptyl, cyclooctyl, and the like.

With respect to the 1,2-dihydropyridine compounds of the invention, the C₃₋₈ cycloalkenyl indicates a cycloalkenyl group composed of 3 to 8 carbon atoms, and examples include cyclopropen-1-yl, cyclopropen-3-yl, cyclobuten-1-yl, cyclobuten-3-yl, 1,3-cyclobutadien-1-yl, cyclopenten-1-yl, cyclopenten-3-yl, cyclopenten-4-yl, 1,3-cyclopentadien-1-yl, 1,3-cyclopentadien-2-yl, 1,3-cyclopentadien-5-yl, cyclohexen-1-yl, cyclohexen-3-yl, cyclohexen-4-yl, 1,3-cyclohexadien-1-yl, 1,3-cyclohexadien-2-yl, 1,3-cyclohexadien-5-yl, 1,4-cyclohexadien-3-yl, 1,4-cyclohexadien-1-yl, cyclohepten-1-yl, cyclohepten-3-yl, cyclohepten-4-yl, cyclohepten-5-yl, 1,3-cyclohepten-2-yl, 1,3-cyclohepten-1-yl, 1,3-cycloheptadien-5-yl, 1,3-cycloheptadien-6-yl, 1,4-cycloheptadien-3-yl, 1,4-cycloheptadien-2-yl, 1,4-cycloheptadien-1-yl, 1,4-cycloheptadien-6-yl, 1,3,5-cycloheptatrien-3-yl, 1,3,5-cycloheptatrien-2-yl, 1,3,5-cycloheptatrien-1-yl, 1,3,5-cycloheptatrien-7-yl, cycloocten-1-yl, cycloocten-3-yl, cycloocten-4-yl, cycloocten-5-yl, 1,3-cyclooctadien-2-yl, 1,3-cyclooctadien-1-yl, 1,3-cyclooctadien-5-yl, 1,3-cyclooctadien-6-yl, 1,4-cyclooctadien-3-yl, 1,4-cyclooctadien-2-yl, 1,4-cyclooctadien-1-yl, 1,4-cyclooctadien-6-yl, 1,4-cyclooctadien-7-yl, 1,5-cyclooctadien-3-yl, 1,5-cyclooctadien-2-yl, 1,3,5-cyclooctatrien-3-yl, 1,3,5-cyclooctatrien-2-yl, 1,3,5-cyclooctatrien-1-yl, 1,3,5-cyclooctatrien-7-yl, 1,3,6-cyclooctatrien-2-yl, 1,3,6-cyclooctatrien-1-yl, 1,3,6-cyclooctatrien-5-yl, 1,3,6-cyclooctatrien-6-yl group, and the like.

With respect to the 1,2-dihydropyridine compounds of the invention, the 5- to 14-membered non-aromatic heterocyclic ring means a mono-cyclic, di-cyclic, or tri-cyclic 5- to 14-membered non-aromatic heterocyclic ring which contains one or more hetero atoms selected from nitrogen, sulfur, and oxygen. Specific examples include pyrrolidinyl, pyrrolyl, piperidinyl, piperazinyl, imidazolyl, pyrazolidyl, imidazolidyl, morpholyl, tetrahydrofuryl, tetrahydropyranyl, pyrrolinyl, dihydrofuryl, dihydropyranyl, imidazolinyl, oxazolinyl, and the like. Further, a group derived from a pyridone ring and a non-aromatic condensed ring (for example, a group derived from a phthalimide ring, a succinimide ring, and the like) are also included in the non-aromatic heterocyclic ring.

With respect to the 1,2-dihydropyridine compounds of the invention, the C₆₋₁₄ aromatic hydrocarbocyclic ring and the aryl mean an aromatic hydrocarbocyclic ring which is composed of 6 to 14 carbon atoms, a mono-cyclic ring, and a condensed di-cyclic, tri-cyclic and the like. Specific examples include phenyl, indenyl, 1-naphthyl, 2-naphthyl, azulenyl, heptalenyl, biphenyl, indathenyl, acenaphthyl, fluorenyl, phenalenyl, phenanthrenyl, anthracenyl, cyclopentacyclooctenyl, benzocyclooctenyl and the like.

With respect to the 1,2-dihydropyridine compounds of the invention, the 5- to 14-membered aromatic heterocyclic ring and the heteroaryl ring mean mono-cyclic, di-cyclic, or tri-cyclic 5- to 14-membered aromatic heterocyclic ring which contain one or more hetero atoms selected from nitrogen, sulfur, and oxygen. Specific examples include (1) aromatic heterocyclic rings containing nitrogen such as pyrrolyl, pyridyl, pyridazinyl, pyrimidinyl, pyrazinyl, triazolyl, tetrazolyl, benzotriazolyl, pyrazolyl, imidazolyl, benzimidazolyl, indolyl, iso-indolyl, indolizinyl, prenyl, indazolyl, quinolyl, iso-quinolyl, quinoliziyl, phthalazyl, naphthylidinyl, quinoxalyl, quinazolinyl, cynnolinyl, pteridinyl, imidazotriazinyl, pyrazinopyridazinyl, acridinyl, phenanthridinyl, carbazolyl, carbazolinyl, perimidinyl, phenanthrolinyl, phenacinyl, imidazopyridinyl, imidazopyrimidinyl, pyrazolopyridinyl, or pyrazolopyridinyl; (2) aromatic heterocyclic rings containing sulfur such as thienyl or benzothienyl; (3) aromatic heterocyclic rings containing oxygen such as furyl, pyranyl, cyclopentapyranyl, benzofuryl or iso-benzofuryl; and (4) aromatic heterocyclic rings containing 2 or more different hetero atoms such as thiazolyl, iso-thiazolyl, benzothiazolyl, benzthiadiazolyl, phenothiazinyl, isoxazolyl, furazanyl, phenoxazinyl, oxazolyl, isoxazoyl, benzoxazolyl, oxadiazolyl, pyrazoloxadiazolyl, imidazothiazolyl, thienofuranyl, furopyrrolyl or pyridoxadinyl.

In another embodiment, the 1,2-dihydropyridine compound used in the methods and compositions described herein is a compound of Formula (III):

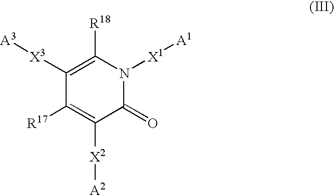

wherein X¹, X², X³, A¹, A², A³, R¹⁷ and R¹⁸ have the same meanings as defined in the above compound of Formula (II).

In another embodiment, the invention provides the compounds of Formula (III) wherein A¹, A² and A³ are each independently an optionally substituted C₆₋₁₄ aromatic hydrocarbon ring or 5- to 14-membered aromatic hetero ring. In another embodiment, the invention provides the compounds of Formula (III) wherein A¹, A² and A³ are each independently phenyl, pyrrolyl, pyridyl, pyridazinyl, pyrimidinyl, pyrazinyl, thienyl, thiazolyl, furyl, naphthyl, quinolyl, iso-quinolyl, indolyl, benzimidazolyl, benzothiazolyl, benzoxazolyl, imidazopyridyl, carbazolyl, cyclopentyl, cyclohexyl, cyclohexenyl, dioxinyl, adamantyl, pyrrolidinyl, piperidinyl, piperazinyl, or morpholyl; wherein each may optionally be substituted. In another embodiment, the invention provides the compounds of Formula (III) wherein A¹, A² and A³ are each independently selected from:

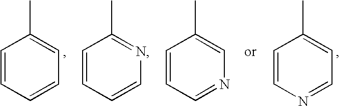

each of which may optionally be substituted. In another embodiment, the invention provides the compounds of Formula (III) wherein the bonding site of the substituent at A¹, A² and A³ are in the α-position of the carbon atom bonding to the group X¹, X² and X³, respectively. In another embodiment, the invention provides the compounds of Formula (III) wherein X¹, X² and X³ are single bonds. In another embodiment, the invention provides the compounds of Formula (III) wherein R⁷ and R¹⁸ are hydrogen.

In one embodiment, the 1,2-dihydropyridine compound used in the methods and compositions described herein is Compound A:

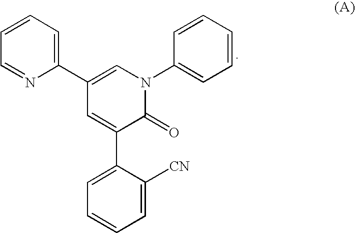

The IUPAC name for Compound A is 2-(2-oxo-1-phenyl-5-pyridin-2-yl-1,2-dihydropyridin-3-yl)benzonitrile. Compound A may also be referred to as 3-(2-cyanophenyl)-5-(2-pyridyl)-1-phenyl-1,2-dihydropyridin-2-one. Compound A is also known as perampanel.

Throughout the specification, the terms “Compound A,” “2-(2-oxo-1-phenyl-5-pyridin-2-yl-1,2-dihydropyridin-3-yl)benzonitrile,” “3-(2-cyanophenyl)-5-(2-pyridyl)-1-phenyl-1,2-dihydropyridin-2-one,” and “perampanel” are intended to include pharmaceutically acceptable salts thereof, stereoisomers thereof, pharmaceutically acceptable salts of stereoisomers thereof, hydrates thereof, hydrates of pharmaceutically acceptable salts thereof, stereoisomers of hydrates thereof, and stereoisomer of hydrates of pharmaceutically acceptable salts thereof. In another embodiment, the terms “Compound A,” “2-(2-oxo-1-phenyl-5-pyridin-2-yl-1,2-dihydropyridin-3-yl)benzonitrile,” “3-(2-cyanophenyl)-5-(2-pyridyl)-1-phenyl-1,2-dihydropyridin-2-one,” and “perampanel” are intended to include pharmaceutically acceptable salts thereof, hydrates thereof, and hydrates of pharmaceutically acceptable salts thereof.

In other embodiments, the 1,2-dihydropyridine compounds that are useful in the methods and compositions of the invention are 3-(2-cyanophenyl)-5-(2-methylsulfonylaminophenyl)-1-phenyl-1,2-dihydropyridin-2-one; 3-(2-chloro-3-pyridyl)-5-(2-pyridyl)-1-phenyl-1,2-dihydropyridin-2-one; 3-(2-cyanophenyl)-5-(2-pyridyl)-1-phenyl-1,2-dihydropyridin-2-one; 3-(2-cyanophenyl)-5-(2-pyridyl)-1-(3-nitrophenyl)-1,2-dihydropyridin-2-one; 3-(2-cyanophenyl)-5-(2-pyridyl)-1-(3-aminophenyl)-1,2-dihydropyridin-2-one; 3-(2-cyanophenyl)-5-(2-pyridyl)-1-(3-methylsulfonylaminophenyl)-1,2-dihydropyridin-2-one; 3-(2-cyanophenyl)-5-(2-pyridyl)-1-(3-methylaminophenyl)-1,2-dihydropyridin-2-one; 3-(2-cyanophenyl)-5-(2-pyridyl)-1-(3-dimethylaminophenyl)-1,2-dihydropyridin-2-one; 3-(2-cyanophenyl)-5-(2-pyridyl)-1-[3-(5-methoxymethyl-2-oxazolidinon-3-yl)-phenyl]-1,2-dihydropyridin-2-one; 3-(2-cyanophenyl)-5-(2-pyridyl)-1-(3-methoxycarbonylphenyl)-1,2-dihydropyridin-2-one; 3-(2-cyanophenyl)-5-(2-pyridyl)-1-(3-methylaminocarbonylphenyl)-1,2-dihydropyridin-2-one; 3-(2-cyano-3-pyridyl)-5-(2-pyridyl)-1-phenyl-1,2-dihydropyridin-2-one; 3-(2-chlorophenyl)-5-(2-pyridyl)-1-(4-hydroxyphenyl)-1,2-dihydropyridin-2-one; 3-(2-chlorophenyl)-5-(2-pyridyl)-1-(4-dimethylaminoethoxyphenyl)-1,2-dihydropyridin-2-one; 3-(2-cyanophenyl)-5-(2-pyridyl)-1-(3-formylphenyl)-1,2-dihydropyridin-2-one; 3-(2-cyanophenyl)-5-(2-pyridyl)-1-(3-hydroxymethylphenyl)-1,2-dihydropyridin-2-one; 3-(2-cyanophenyl)-5-(2-pyridyl)-1-(3-cyanomethylphenyl)-1,2-dihydropyridine-2-one; 3-(2-cyanophenyl)-5-(2-pyridyl)-1-(3-acetylaminomethylphenyl)-1,2-dihydropyridin-2-one; 3-(2-cyanophenyl)-5-(2-pyridyl)-1-(3-methylsulfonylaminomethylphenyl)-1,2-dihydropyridin-2-one; 3-(2-cyanophenyl)-5-(2-pyridyl)-1-(3-acetoxymethylphenyl)-1,2-dihydropyridin-2-one; 3-(2-cyanophenyl)-5-(2-pyridyl)-1-(4-methylthiophenyl)-1,2-dihydropyridin-2-one; 3-(2-cyanophenyl)-5-(2-pyridyl)-1-(4-methylsulfonylpheny-1)-1,2-dihydropyridin-2-one; 3-(2-cyanophenyl)-5-(2-formylthiophen-3-yl)-1-phenyl-1,2-dihydropyridin-2-one; 3-(2-cyanophenyl)-5-(2-diethylaminomethylthiophen-3-yl)-1-phenyl-1,2-dihydropyridin-2-one; 3-(2-cyanophenyl)-5-(2-hydroxymethylthiophen-3-yl)-1-phenyl-1,2-dihydropyridine-2-one; 3-(2-cyanophenyl)-5-(2-pyridyl)-1-benzyl-1,2-dihydropyridin-2-one; 3-(2-cyanophenyl)-5-(2-pyridyl)-1-(3-pyridyl)-1,2-dihydropyridin-2-one; 3-(2-cyanophenyl)-5-phenyl-(2-pyridyl)-1,2-dihydropyridin-2-one; 3-(2-cyanophenyl)-1,5-diphenyl-1,2-dihydropyridin-2-one; 3-(2-cyanophenyl)-5-(2-methoxyphenyl)-1-phenyl-1,2-dihydropyridin-2-one; 3-(2-cyanophenyl)-5-(3,4-dimethoxyphenyl)-1-phenyl-1,2-dihydropyridin-2-one; 3-(2-cyanophenyl)-5-(thiophen-3-yl)-1-phenyl-1,2-dihydropyridin-2-one; 3-(2-cyanophenyl)-5-(2-fluorophenyl)-1-phenyl-1,2-dihydropyridin-2-one; 3-(2-cyanophenyl)-5-(thiophen-2-yl)-1-phenyl-1,2-dihydropyridin-2-one; 3-(2-cyanophenyl)-5-(3-furfuryl)-1-phenyl-1,2-dihydropyridin-2-one; 3-(2-cyanophenyl)-5-(2-furfuryl)-1-phenyl-1,2-dihydropyridin-2-one; 3-(2-chlorophenyl)-5-(2-pyridyl)-1-phenyl-1,2-dihydropyridin-2-one; 3-(2-methoxycarbonylphenyl)-5-(2-pyridyl)-1-phenyl-1,2-dihydropyridin-2-one; 3-phenyl-5-(2-pyridyl)-1-phenyl-1,2-dihydropyridin-2-one; 3-(2-fluorophenyl)-5-(2-pyridyl)-1-phenyl-1,2-dihydropyridin-2-one; 3-(2-chlorophenyl)-5-(2-pyridyl)-1-(3-methoxyphenyl)-1,2-dihydropyridin-2-one; 3-(2-fluoro-3-pyridyl)-5-(2-pyridyl)-1-phenyl-1,2-dihydropyridin-2-one; 3-(4-methoxy-3-pyridyl)-5-(2-pyridyl)-1-phenyl-1,2-dihydropyridin-2-one; 3-(2-fluoro-3-pyridyl)-5-(2-pyridyl)-1-(3-methoxyphenyl)-1,2-dihydropyridin-2-one; 3-(2-fluoro-3-pyridyl)-5-(2-pyridyl)-1-(3-fluorophenyl)-1,2-dihydropyridin-2-one; 3-(2-cyanophenyl)-5-(2-pyridyl)-1-(4-fluorophenyl)-1,2-dihydropyridin-2-one; 3-(2-cyanophenyl)-5-(2-pyridyl)-1-(3-fluorophenyl)-1,2-dihydropyridin-2-one; 3-(2-cyanophenyl)-5-(2-pyridyl)-1-(4-methoxyphenyl)-1,2-dihydropyridin-2-one; 3-(2-cyanophenyl)-5-(2-pyridyl)-1-(3-methoxy-phenyl)-1,2-dihydropyridin-2-one; 3-phenyl-5-(2-pyridyl)-1-(3-fluorophenyl-)-1,2-dihydropyridin-2-one; 3-(2-chlorophenyl)-5-(2-pyridyl)-1-(4-fluorophenyl)-1,2-dihydropyridin-2-one; 3-(2-cyanophenyl)-5-(2-pyridyl)-1-(4-formylphenyl)-1,2-dihydropyridin-2-one; 3-(2-cyanophenyl)-5-(2-pyridyl)-1-(2-formylphenyl)-1,2-dihydropyridin-2-one; 3-(2-cyanophenyl)-5-(2-pyridyl)-1-(3-chlorophenyl)-1,2-dihydropyridin-2-one; 3-(2-cyanophenyl)-5-(2-pyridyl)-1-(3-tolyl)-1,2-dihydropyridin-2-one; 3-(2-cyanophenyl)-5-(2-pyridyl)-1-(3-trifluoromethylphenyl)-1,2-dihydropyridin-2-one; 3-(2-cyanophenyl)-5-(2-pyridyl)-1-(thiophen-3-yl)-1,2-dihydropyridin-2-one; 3-(2-cyanophenyl)-5-(2-pyridyl)-1-(3-furfuryl)-1,2-dihydropyridin-2-one; 3-(2-cyanophenyl)-5-(2-pyridyl)-1-(4-tolyl)-1,2-dihydropyridin-2-one; 3-(2-cyanophenyl)-5-(2-pyridyl)-1-(4-trifluoromethylphenyl)-1,2-dihydropyridin-2-one; 3-(2-cyanophenyl)-5-(2-pyridyl)-1-(2-methoxypyridin-5-yl)-1,2-dihydropyridin-2-one; 3-(2-cyanophenyl)-5-(2-pyridyl)-1-(pyrimidin-5-yl)-1,2-dihydropyridin-2-one; 3-(2-cyanophenyl)-5-(2-pyridyl)-1-(3-benzyloxymethylpyridin-5-yl)-1,2-dihydropyridin-2-one; 3-(2-cyanophenyl)-5-(2-pyridyl)-1-(2-ethylthiopyridin-5-yl)-1,2-dihydropyridin-2-one; 3-(2-cyanophenyl)-5-(2-pyridyl-)-1-(4-pyridyl)-1,2-dihydropyridin-2-one; 3-(2-cyanophenyl)-5-(2-pyridyl)-1-(3-methoxypyridin-5-yl)-1,2-dihydropyridin-2-one; 3-(2-cyanophenyl)-5-(2-pyridyl)-1-(2-chloropyridin-5-yl)-1,2-dihydropyridin-2-one; 3-(2-cyanophenyl)-5-(2-pyridyl)-1-(2-fluoropyridin-5-yl)-1,2-dihydropyridin-2-one; 3-(2-cyanophenyl)-5-(2-pyridyl)-1-(2-methoxyphenyl)-1,2-1-dihydropyridin-2-one; 3-phenyl-5-(2-pyridyl)-1-(3-pyridyl)-1,2-dihydropyridin-2-one; 3-(2-chlorophenyl)-5-(2-pyridyl)-1-(3-pyridyl)-1,2-dihydropyridin-2-one; 3-(thiophen-3-yl)-5-(2-pyridyl)-1-(3-pyridyl)-1,2-dihydropyridin-2-one; 3-(2,6-dimethylphenyl)-5-(2-pyridyl)-1-(3-pyridyl)-1,2-dihydropyridin-2-one; 3-(2-cyanothiophen-3-yl)-5-(2-pyridyl)-1-(3-pyridyl)-1,2-dihydropyridin-2-one; 3-(2-fluoro-3-pyridyl)-5-(2-pyridyl)-1-(3-pyridyl)-1,2-dihydropyridin-2-one; 3-(2-chlorophenyl)-5-(2-pyridyl)-1-(3-hydroxyphenyl)-1-,2-dihydropyridin-2-one; 3-(2-chlorophenyl)-5-(2-pyridyl)-1-(3-dimethylaminoethoxyphenyl)-1,2-dihydropyridin-2-one; 3-(2-chlorophenyl)-5-(2-pyridyl)-1-(3-dimethylaminopropoxyphenyl)-1,2-dihydropyridin-2-one; 3-(2-cyanophenyl)-5-(2-pyridyl)-1-(2-hydroxymethylphenyl)-1,2-dihydropyridin-2-one; 3-(2-cyanophenyl)-5-(2-pyridyl)-1-(4-cyanomethylphenyl)-1,2-dihydropyridin-2-one; 3-(2-cyanophenyl)-5-(2-pyridyl)-1-(2-cyanomethylphenyl)-1,2-dihydropyridin-2-one; 3-(2-cyanophenyl)-5-(6-diethylaminomethyl-2-pyridyl)-1-phenyl-1,2-dihydropyridin-2-one; 3-(2-cyanophenyl)-1-phenyl-5-(2-pyrimidinyl)-1,2-dihydropyridin-2-one; 3-(2-hydroxypyridin-6-yl)-1-phenyl-5-(2-pyridyl)-1,2-dihydropyridin-2-one; 1-(2-aminobenzothiazol-6-yl)-3-(2-cyanophenyl)-5-(2-pyridyl)-1,2-dihydropyridin-2-one; 3-(2-cyanophenyl)-5-(2-pyridyl)-1-(1-benzyl-1,2,3,6-tetrahydropyridin-5-yl)-1,2-dihydropyridin-2-one; 3-[2-(5-methyl-1,2,4-oxadiazol-3-yl)phenyl]-1-phenyl-5-(2-pyridyl)-1,2-dihydropyridin-2-one; 3-(2-cyanophenyl)-5-(6-methylpyridin-2-yl)-1-phenyl-1,2-dihydropyridin-2-one; 3-(2-cyanophenyl)-5-(5-methylpyridin-2-yl)-1-phenyl-1,2-dihydropyridin-2-one; 3-(2-cyanophenyl)-5-(3-hydroxypyridin-2-yl)-1-phenyl-1,2-dihydropyridin-2-one; 3-(2-cyanophenyl)-1-phenyl-5-(2-thiazolyl)-1,2-dihydropyridin-2-one; 3-(2-cyanophenyl)-5-(2-methoxypyridin-6-yl)-1-phenyl-1,2-dihydropyridin-2-one; 1-(4-aminophenyl)-3-(2-cyanophenyl)-5-(2-pyridyl)-1,2-dihydropyridin-2-one; 1-(3-aminophenyl)-3-(2-cyanophenyl)-5-(2-pyrimidinyl)-1,2-dihydropyridin-2-one; 3-(2-cyanophenyl)-5-(2-pyridyl)-1-(2-aminotoluen-4-yl)-1-,2-dihydropyridin-2-one; 3-(2-cyanophenyl)-1-[3-(dimethylamino ethoxy)phenyl]-5-(2-pyridyl)-1,2-dihydropyridin-2-one; 3-(2-cyanophenyl)-1-[3-(piperidinoethoxy)phenyl]-5-(2-pyridyl)-1,2-dihydropyridin-2-one; 3-(2-cyanophenyl)-1-[3-(pyrrolidinoethoxy)phenyl]-5-(2-pyridyl)-1,2-dihydropyridin-2-one; 3-(2-cyanophenyl)-1-[3-(diisoproylaminoethoxy)phenyl]-5-(2-pyridyl)-1,2-dihydropyridin-2-one; 3-(2-cyanophenyl)-1-[3-(4-piperidinobutyl-1-oxy)phenyl]-5-(2-pyridyl)-1,2-dihydropyridin-2-one; 3-(2-cyanophenyl)-1-(4-nitrophenyl)-5-(2-pyridyl)-1,2-dihydropyridin-2-one; 1-phenyl-5-(2-pyridyl)-3-(2-thiazolyl)-1,2-dihydropyridin-2-one; 3-(2-cyanophenyl)-1-(3-pyridyl)-5-(2-pyrimidinyl)-1,2-dihydropyridin-2-one; 3-(2-fluoropyridin-3-yl)-1-phenyl-5-(2-pyrimidinyl)-1,2-dihydropyridin-2-one; 3-(2-cyanopyridin-3-yl)-1-phenyl-5-(2-pyrimidinyl)-1,2-dihydropyridin-2-one; 3-(2-cyanophenyl)-1-(3-nitrophenyl)-5-(2-pyrimidinyl)-1,2-dihydropyridin-2-one; 3-(2-nitrophenyl)-1-phenyl-5-(2-pyridyl)-1,2-dihydropyridin-2-one; 3-(2-formylthiophen-3-yl)-5-(2-pyridyl)-1-phenyl-1,2-dihydropyridin-2-one; 3-(2-cyanophenyl)-5-(2-pyridyl)-1-(2-naphthyl)-1,2-dihydropyridin-2-one; 3-(2-cyanophenyl)-5-(2-pyridyl)-1-(1-naphthyl)-1,2-dihydropyridin-2-one; 5-(2-aminopyridin-6-yl)-3-(2-cyanophenyl)-1-phenyl-1,2-dihydropyridin-2-one; 5-(2-bromopyridin-6-yl)-3-(2-cyanophenyl)-1-phenyl-1,2-dihydropyridin-2-one; 3-(2-cyanophenyl)-5-(2-morphorinopyridin-6-yl)-1-phenyl-1,2-dihydropyridin-2-one; 3-(2-cyanophenyl)-1-(3-hydroxyphenyl)-5-(2-pyridyl)-1,2-dihydropyridin-2-one; 3-(2-cyanophenyl)-1-[3-(4-piperidyloxy)]phenyl-5-(2-pyridyl)-1,2-dihydropyridin-2-one; 1-[3-(N-acetylpiperidin-4-yl-oxy)phenyl]-3-(2-cyanophenyl)-5-(2-pyridyl)-1,2-dihydropyridin-2-one; 3-(2-cyanophenyl)-1-{3-[1-(methanesulfonyl)piperidin-4-yl-oxy]phenyl}-5-(2-pyridyl)-1,2-dihydropyridin-2-one; 1-[3-(N-methylpiperidin-4-yl-oxy)phenyl]-3-(2-cyanophenyl)-5-(2-pyridyl)-1,2-dihydropyridin-2-one; 3-(6-chloro-1H-benzimidazol-2-yl)-5-(2-pyridyl)-1-phenyl-1,2-dihydropyridin-2-one; 3-(2-cyanophenyl)-5-(2-pyridyl)-1-(2-nitrotoluen-4-yl)-1,2-dihydropyridin-2-one; 3-(2-cyanothiophen-3-yl)-5-(2-pyridyl)-1-phenyl-1,2-dihydropyridin-2-one; 3-[2-(5-oxazolyl)phenyl]-1-phenyl-5-(2-pyridyl)-1,2-dihydropyridin-2-one; 3-[2-(5-oxazolyl)thiophen-3-yl]-1-phenyl-5-(2-pyridyl)-1,2-dihydropyridin-2-one; and 3-(2-ethoxycarbonylvinylthiophen-3-yl)-5-(2-pyridyl)-1-phenyl-1,2-dihydropyridin-2-one.

The 1,2-dihydropyridine compounds and methods for making the 1,2-dihydropyridine compounds are described in U.S. Pat. No. 6,949,571, US Publication No. 2004/0023973, and PCT Publication No. WO 03/047577, WO 04/009553, WO 06/004100, WO 06/004107, WO 07/072,868, WO 07/072,869, WO 07/126,060, and WO 08/093,392, the disclosures of which are incorporated by reference herein in their entirety.

Methods for administering, dosing, and making other AMPA receptor antagonists such as quinoxalinedione aminoalkylphosphonates are described, for example, in WO 2005/094797 and WO 98/17672.

Zonisamide is commercially available or can be prepared by methods well known in the literature. Zonisamide is available as EXCEGRAN® in the form of tablets or powder (Dainippon Sumitomo Pharma, Japan) and as ZONEGRAN® (Eisai, Inc., Teaneck, N.J.) in the United States. Zonisamide may be produced according to a method described, for example, in Japanese Examined Application No. 60-33114, Japanese Examined Application No. 61-59288 and U.S. Pat. No. 4,172,896.

Zonisamide may be used in the same manner in its salt form. Salts refer to pharmaceutically acceptable salts known in the art, and not particularly limited as long as they form pharmaceutically acceptable salts with zonisamide. Specifically, examples include quaternized amine salts, alkali metal salts (e.g., sodium salt, potassium salt and lithium salt) and alkaline earth metal salts (e.g., magnesium salt and calcium salt).

In other embodiments, the invention provides pharmaceutical compositions comprising a therapeutically effective amount of: (i) at least one AMPA receptor antagonist, (ii) zonisamide, and (iii) at least one pharmaceutically acceptable excipient. The invention also provides combinations comprising a therapeutically effective amount of: (i) at least one AMPA receptor antagonist and (ii) zonisamide; wherein the compounds may be administered separately (e.g., simultaneously, sequentially) to a patient to treat the diseases or disorders described. The invention provides kits (e.g., commercial packages) comprising a therapeutically effective amount of: (i) at least one AMPA receptor antagonist, (ii) zonisamide; and (iii) instructions for the simultaneous, separate or sequential use of (i) and (ii) in the treatment of the diseases and disorders described herein. The AMPA receptor antagonist can be any described herein. For example, the 1,2-dihydropyridine compound can be a compound of Formula (I), a compound of Formula (II), a compound of Formula (III), or Compound A. In one embodiment, the invention provides pharmaceutical compositions comprising a therapeutically effective amount of: (i) zonisamide; (ii) 3-(2-cyanophenyl)-5-(2-pyridyl)-1-phenyl-1,2-dihydropyridin-2-one; and (iii) at least one pharmaceutically acceptable excipient.

The invention provides methods for the treatment and/or prophylaxis of epilepsy, one or more symptoms of epilepsy in a patient in need thereof by administering a therapeutically effective amount of: (a) at least one AMPA receptor antagonist, and (b) zonisamide or a pharmaceutically acceptable salt thereof. The methods for the treatment of epilepsy and one or more symptoms of epilepsy include (i) methods for reducing the frequency of epilepsy, or one or more symptoms of epilepsy, (ii) methods for reducing the severity of epilepsy, or one or more symptoms of epilepsy, (iii) methods for reducing the duration of epilepsy, or one or more symptoms of epilepsy, (iv) methods for reducing the frequency and severity of epilepsy, or one or more symptoms of epilepsy, (v) methods for reducing the frequency and duration of epilepsy, or one or more symptoms of epilepsy, (vi) methods for reducing the severity and duration of epilepsy, or one or more symptoms of epilepsy, and (vii) methods for reducing the frequency, severity and duration of epilepsy, or one or more symptoms of epilepsy. The AMPA receptor antagonist and, optionally, zonisamide or a pharmaceutically acceptable salt thereof, can be administered separately to the patient or may be administered in the form of a pharmaceutical composition.

The dosage form of the formulation included in the combination, kit and/or pharmaceutical composition of the invention is not particularly limited. The combination, kit and/or pharmaceutical composition of the invention is useful as a combination, kit and/or a pharmaceutical composition for treating neuropathic pain; for the prophylaxis of neuropathic pain; and for delaying the onset of neuropathic pain.

The combination, kit and/or pharmaceutical composition of the invention may be used as a drug for treating neuropathic pain; for the prophylaxis of neuropathic pain; and for delaying the onset of neuropathic pain.

The combination, kit and/or pharmaceutical composition of the invention may be administered to a patient.

The combination, kit and/or pharmaceutical composition of the invention may be used through oral or parenteral administration. When the combination, kit and/or pharmaceutical composition of the invention is used, the given dose of the compound of the invention differs depending on the degree of the symptom, age, sex, weight and sensitivity difference of the patient, administration mode, administration period, administration interval, nature, prescription and the type of the pharmaceutical formulation, and the type of the active element.

The pharmaceutical composition of the invention may be made into various forms, for example, into solid oral formulations, injectable solution or the like.

The AMPA receptor antagonists and zonisamide can be administered orally, topically, parenterally, by inhalation (nasal or oral), or rectally in dosage unit formulations containing conventional nontoxic pharmaceutically acceptable carriers, adjuvants, and vehicles as desired. The term parenteral includes subcutaneous, intravenous, intramuscular, intrathecal, intrasternal injection, or infusion techniques.

The daily dose of the AMPA receptor antagonists of the invention (e.g., 3-(2-cyanophenyl)-5-(2-pyridyl)-1-phenyl-1,2-dihydropyridin-2-one) is usually 30 μg to 10 g/day; 100 μg to 5 g/day; or 100 μg to 100 mg/day, in the case of oral administration. For administration by injection, the daily dose is usually 30 μg to 1 g/day; 100 μg to 500 mg/day; or 100 mg to 30 mg/day. The compounds are administered once daily or in several portions a day. When used in the context of a dosage amount, the numerical weight refers to the weight of the 1,2-dihydropyridine, exclusive of any salt, counterion, hydrate, and the like. Therefore to obtain the equivalent of 500 mg of 3-(2-cyanophenyl)-5-(2-pyridyl)-1-phenyl-1,2-dihydropyridin-2-one, it would be necessary to use more than 500 mg of a pharmaceutically acceptable salt and/or hydrate of the compound, due to the additional weight of the pharmaceutically acceptable salt and/or hydrate.

The daily dose of zonisamide of the invention is usually 0.1 mg/day to 3000 mg/day; 1 mg/day to 600 mg/day; or 5 mg/day to 300 mg/day. In other embodiment, the daily dose is from 0.01 mg/day to 500 mg/day; from 0.1 mg/day to 100 mg/day; or from 0.1 mg/day to 30 mg/day. The compounds are administered once daily or in several portions a day. When used in the context of a dosage amount, the numerical weight refers to the weight of zonisamide, exclusive of any salt, and the like.

When administered to a child, the dose may possibly be lower than that for an adult. The actual method for administration may fluctuate widely and may depart from the preferred method described herein.

Any other compounds described herein may be administered in doses well known in the art by reference, for example, to The Physician's Desk Reference, to patents describing doses for the compounds, and to journal articles describing doses for the compounds.

In one embodiment, the mode of administration is by injection, such as subcutaneous injection, intramuscular injection, intravenous injection, or intra-arterial injection. Injectable preparations, for example, sterile injectable aqueous or oleaginous suspensions can be formulated according to the art using suitable dispersing or wetting agents, suspending agents (e.g., methylcellulose, Polysorbate 80, hydroxyethylcellulose, acacia, powdered tragacanth, sodium carboxymethylcellulose, polyoxytehylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like), stabilizers (e.g., sodium sulfite and sodium metasulfite; examples of the preservative include methyl parahydroxybenzoate, ethyl parahydroxybenzoate, sorbic acid, phenol, cresol, chlorocresol, and the like), tonicity agents and preservatives. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. In addition, sterile, fixed oils are conventionally used as a solvent or suspending medium. For this purpose any bland fixed oil can be used including synthetic mono- or diglycerides, in addition, fatty acids, such as oleic acid, can be used in the preparation of injectables. The preparations can be lyophilized by methods known in the art.

In order to prepare a solid oral formulation, an excipient, and if necessary, a binder, disintegrant, lubricant, colorant, a flavoring agent and the like are added to the principal agent, and then made into a tablet, a coated tablet, granule, fine granule, dispersant, a capsule or the like according to a conventional method.

For example, lactose, cornstarch, sucrose, glucose, sorbit, crystalline cellulose, silicon dioxide or the like may be used as the excipient; for example, polyvinyl alcohol, ethyl cellulose, methyl cellulose, gum arabic, hydroxypropyl cellulose, hydroxypropylmethyl cellulose or the like may be used as the binder; for example, magnesium stearate, talc, silica or the like may be used as the lubricant; those that are allowed to be added to drugs may be used as the colorant; and for example, cocoa powder, menthol, aromatic acid, peppermint oil, camphor, cinnamon powder or the like may be used as the flavoring agent. Of course, if necessary, these tablets and granule may be coated appropriately with sugar coating, gelatin coating or else.

Solid dosage forms for oral administration can include chewing gum, capsules, tablets, sublingual tablets, powders, granules, and gels. In such solid dosage forms, the active compound can be admixed with one or more inert diluents such as lactose or starch. As is normal practice, such dosage forms can also comprise other substances including lubricating agents such as magnesium stearate. In the case of capsules, tablets, and pills, the dosage forms can also comprise buffering agents. The tablets can be prepared with enteric or film coatings, preferably film coatings.

To make tablets, the compounds can be admixed with pharmaceutically acceptable carriers known in the art such as, for example, vehicles (e.g., lactose, white sugar, mannitol, glucose, starches, calcium carbonate, crystalline cellulose, silicic acid, and the like), binders (e.g., water, ethanol, myranol, glucose solution, starch solution, gelatin solution, polyvinylpyrrolidone, and the like), disintegrators (e.g., dry starch, sodium, alginate, sodium hydrogen carbonate, calcium carbonate, polyoxyethylene sorbitan fatty acid esters, sodium laurylsulfate, stearic monoglyceride, starches, lactose, and the like), absorption promoters (e.g., quaternary ammonium base, sodium laurylsulfate, and the like), wetting agents (e.g. glycerin, starches, and the like), lubricants (e.g., stearates, polyethylene glycol, and the like), and flavoring agents (e.g., sweeteners). The tablets can be in the form of a conventional tablet, a molded tablet, a wafer and the like.

Sublingual administration refers to the administration in the mouth (e.g., under the tongue, between the cheek and gum, between the tongue and roof of the mouth). The highly vascular mucosal lining in the mouth is a convenient location for the compounds to be administered into the body.

In other embodiments, the solid dosage form can be packaged as granules or a powder in a pharmaceutically acceptable carrier, where the granules or powder are removed from the packaging and sprinkled on food or mixed with a liquid, such as water or juice, or where the granules are inserted into capsules. In this embodiment, the compounds described herein can be mixed with flavoring or sweetening agents. The packaging material can be plastic, coated paper, or any material that prevents water or moisture from reaching the granules and/or powder.

Liquid dosage forms for oral administration can include pharmaceutically acceptable emulsions, solutions, sublingual solutions, suspensions, and syrups containing inert diluents commonly used in the art, such as water. Such compositions can also comprise adjuvants, such as wetting agents, emulsifying and suspending agents, and sweetening, flavoring, and perfuming agents. To make sublingual solutions, the compounds can be admixed with various carriers, excipients, pH adjusters, and the like (e.g., water, sugar, lactic acid, acetic acid, fructose, glucose, saccharin, polyethylene glycol, propylene glycol, alcohol, bentonite, tragacanth, gelatin, alginates, aspartame, sorbitol, methylparaben, propylparaben, sodium benzoate, artificial flavoring and coloring agents).

Each of the patents, patent applications, and publications cited herein are incorporated by reference herein in their entirety.

It will be apparent to one skilled in the art that various modifications can be made to the invention without departing from the spirit or scope of the appended claims.

1. A pharmaceutical composition comprising:
(A) an AMPA receptor antagonist, a pharmaceutically acceptable salt thereof, a hydrate thereof, or a hydrate of a pharmaceutically acceptable salt thereof;
(B) zonisamide or a pharmaceutically acceptable salt thereof; and
(C) one or more pharmaceutically acceptable carries.

2. The pharmaceutical composition of claim 1, wherein the AMPA receptor antagonist, pharmaceutically acceptable salt thereof, hydrate thereof, or the hydrate of the pharmaceutically acceptable salt thereof is a compound of Formula (III), a pharmaceutically acceptable salt thereof, a hydrate thereof, or a hydrate of a pharmaceutically acceptable salt thereof, wherein the compound of Formula (III) is: 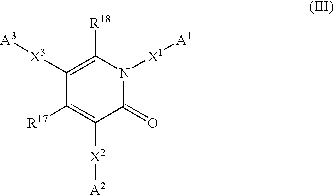
wherein X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶); R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

3. The pharmaceutical composition of claim 1, wherein the AMPA receptor antagonist, pharmaceutically acceptable salt thereof, hydrate thereof, or hydrate of a pharmaceutically acceptable salt thereof is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

4. The pharmaceutical composition of claim 1, wherein the composition is used for treating epilepsy or one or more symptoms of epilepsy.

5. The pharmaceutical composition of claim 1, wherein the composition is used for treating partial seizure or one or more symptoms of partial seizure.

6. The pharmaceutical composition of claim 5, wherein the partial seizure is a simple partial seizure, complex partial seizure, or a partial seizure secondarily generalized.

7. The pharmaceutical composition of claim 1, wherein the composition is used for treating generalized seizure or one, or more symptoms of generalized seizure.

8. The pharmaceutical composition of claim 7, wherein the generalized seizure is an absence seizures, myoclonic seizures, clonic seizures, tonic seizures, tonic-clonic seizures or atonic seizures.

9. The pharmaceutical composition of claim 1, wherein the composition is adapted to be associated with a treatment regimen.

10. A combination comprising:
(A) an AMPA receptor antagonist, a pharmaceutically acceptable salt thereof, a hydrate thereof, or a hydrate of a pharmaceutically acceptable salt thereof; and
(B) zonisamide or a pharmaceutically acceptable salt thereof.

11. The combination of claim 10, wherein the AMPA receptor antagonist, pharmaceutically acceptable salt thereof, hydrate thereof, or the hydrate of the pharmaceutically acceptable salt thereof is a compound of Formula (III), a pharmaceutically acceptable salt thereof, a hydrate thereof, or a hydrate of a pharmaceutically acceptable salt thereof; wherein the compound of Formula (III) is: 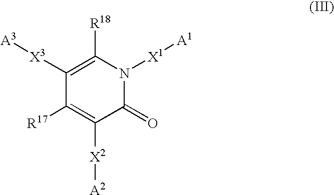
wherein X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶); R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

12. The combination of claim 10, wherein the AMPA receptor antagonist, pharmaceutically acceptable salt thereof, hydrate thereof, or hydrate of a pharmaceutically acceptable salt thereof is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

13. The combination of claim 10, wherein (A) and (B) are administered separately to a patient or are administered to a patient in the form of a pharmaceutical composition.

14. The combination of claim 10, wherein the combination is used for treating epilepsy or one or more symptoms of epilepsy.

15. The combination of claim 10, wherein the combination is used for treating partial seizure or one or more symptoms of partial seizure.

16. The combination of claim 15, wherein the partial seizure is a simple partial seizure, complex partial seizure, or a partial seizure secondarily generalized.

17. The combination of claim 10, wherein the combination is used for treating generalized seizure or one or more symptoms of generalized seizure.

18. The combination of claim 17, wherein the generalized seizure is an absence seizures, myoclonic seizures, clonic seizures, tonic seizures, tonic-clonic seizures or atonic seizures.

19. The combination of claim 10, wherein the combination is adapted to be associated with a treatment regimen.

20. Use of compounds (A) and (B) for producing a pharmaceutical composition in the treatment of epilepsy or one or more symptoms of epilepsy, wherein (A) and (B) are:
(A) an AMPA receptor antagonist, a pharmaceutically acceptable salt thereof, a hydrate thereof, or a hydrate of a pharmaceutically acceptable salt thereof, and
(B) zonisamide or a pharmaceutically acceptable salt thereof.

21. Use of compounds (A) and (B) for producing a pharmaceutical composition in the treatment of partial seizure or one or more symptoms of partial seizure, wherein (A) and (B) are:
(A) an AMPA receptor antagonist, a pharmaceutically acceptable salt thereof, a hydrate thereof, or a hydrate of a pharmaceutically acceptable salt thereof; and
(B) zonisamide or a pharmaceutically acceptable salt thereof.

22. The use of claim 21, wherein the partial seizure is a simple partial seizure, complex partial seizure, or a partial seizure secondarily generalized.

23. Use of compounds (A) and (B) for producing a pharmaceutical composition in the treatment of generalized seizure or one or more symptoms of generalized seizure, wherein (A) and (B) are:
(A) an AMPA receptor antagonist, a pharmaceutically acceptable salt thereof, a hydrate thereof, or a hydrate of a pharmaceutically acceptable salt thereof; and
(B) zonisamide or a pharmaceutically acceptable salt thereof.

24. The use of claim 23, wherein the generalized seizure is an absence seizures, myoclonic seizures, clonic seizures, tonic seizures, tonic-clonic seizures or atonic seizures.

25. The use of any one of claims 20 to 24, wherein the AMPA receptor antagonist, pharmaceutically acceptable salt thereof, hydrate thereof, or the hydrate of the pharmaceutically acceptable salt thereof is a compound of Formula (III), a pharmaceutically acceptable salt thereof, a hydrate thereof, or a hydrate of a pharmaceutically acceptable salt thereof; wherein the compound of Formula (III) is: 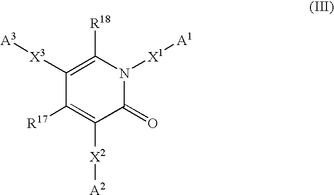
wherein X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)ₚ, —S(O)_{q}—CH₂—, —CH₂—O—, —O—CH₂—, —N(R¹³)—CO—N(R¹⁴)— or —N(R¹⁵)—CS—N(R¹⁶); R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

26. The use of any one of claims 20 to 24, wherein the AMPA receptor antagonist, pharmaceutically acceptable salt thereof, hydrate thereof, or hydrate of a pharmaceutically acceptable salt thereof is 3-(2-cyanophenyl)-5-(2-pyridyl)-1-phenyl-1,2-dihydropyridin-2-one, a pharmaceutically acceptable salt thereof, a hydrate thereof, or a hydrate of a pharmaceutically acceptable salt thereof.

27. The use of any one of claims 20 to 24, wherein (A) and (B) are administered separately to a patient or are administered to a patient in the form of a pharmaceutical composition.

28. The use of any one of claims 20 to 24, wherein the treatment is part of a treatment regimen.

29. Compounds (A) and (B) for use in the treatment of epilepsy or one or more symptoms of epilepsy, wherein (A) and (B) are:
(A) an AMPA receptor antagonist, a pharmaceutically acceptable salt thereof, a hydrate thereof, or a hydrate of a pharmaceutically acceptable salt thereof; and
(B) zonisamide or a pharmaceutically acceptable salt thereof.

30. Compounds (A) and (B) for use in the treatment of partial seizure or one or more symptoms of partial seizure, wherein (A) and (B) are:
(A) an AMPA receptor antagonist, a pharmaceutically acceptable salt thereof, a hydrate thereof, or a hydrate of a pharmaceutically acceptable salt thereof; and
(B) zonisamide or a pharmaceutically acceptable salt thereof.

31. The compound of claim 30, wherein the partial seizure is a simple partial seizure, complex partial seizure, or a partial seizure secondarily generalized.

32. Compounds (A) and (B) for use in the treatment of generalized seizure or one or more symptoms of generalized seizure, wherein (A) and (B) are:
(A) an AMPA receptor antagonist, a pharmaceutically acceptable salt thereof, a hydrate thereof, or a hydrate of a pharmaceutically acceptable salt thereof; and
(B) zonisamide or a pharmaceutically acceptable salt thereof.

33. The compound of claim 32, wherein the generalized seizure is an absence seizures, myoclonic seizures, clonic seizures, tonic seizures, tonic-clonic seizures or atonic seizures.

34. The compound of any one of claims 29 to 33, wherein the treatment is part of a treatment regimen.

35. A kit comprising the pharmaceutical composition of any one of claims 1 to 9 or the combination of any one of claims 10 to 19.

36. The kit of claim 35, wherein the kit is adapted to be associated with a treatment regimen.

37. A method for treating epilepsy or one or more symptoms of epilepsy comprising administering to a patient in need thereof a therapeutically effective amount of the pharmaceutical composition of any one of claims 1 to 9 or a therapeutically effective amount of the combination of any one of claims 10 to 19.

38. A method for treating partial seizure or one or more symptoms of partial seizure comprising administering to a patient in need thereof a therapeutically effective amount of the pharmaceutical composition of any one of claims 1 to 9 or a therapeutically effective amount of the combination of any one of claims 10 to 19.

39. The method of claim 38, wherein the partial seizure is a simple partial seizure, complex partial seizure, or a partial seizure secondarily generalized.

40. A method for treating generalized seizure or one or more symptoms of generalized seizure comprising administering to a patient in need thereof a therapeutically effective amount of the pharmaceutical composition of any one of claims 1 to 9 or a therapeutically effective amount of the combination of any one of claims 10 to 19.

41. The method of claim 40, wherein the generalized seizure is an absence seizures, myoclonic seizures, clonic seizures, tonic seizures, tonic-clonic seizures or atonic seizures.

42. The method of any one of claims 37 to 41, wherein the treatment is part of a treatment regimen and the administration involves a series of administrations.
